(19) United States
(12) Patent Application Publication
(10) Pub. No.: US 2010/0028322 A1
(43) Pub. Date: Feb. 4, 2010

(54) ANTIMETASTATIC EFFECT ON HUMAN CELL DISORDERS

(75) Inventor: Pablo Rodriguez, Cordoba (AR)

Correspondence Address: STANDLEY LAW GROUP LLP, 6300 Riverside Drive, Dublin, OH, 43017, US

(73) Assignee: THERAPICON SRL, Milan (IT)

(21) Appl. No.: 12/445,262

(22) PCT Filed: Oct. 9, 2007
(86) PCT No.: PCT/EP07/60729

§ 371 Date: Apr. 10, 2009

(30) Foreign Application Priority Data
Oct. 11, 2006 (IT) MI2006A001950

(51) Int. Cl. A61K 38/47; A61P 35/00

(52) U.S. Cl. 424/94.61

(57) ABSTRACT

Use of recombinant human lysozyme in the preparation of a medicament for controlling life-threatening diseases associated with abnormal cell proliferation and migration, such as cancer metastasis, by administering to a subject in need thereof therapeutically effective doses of recombinant human lysozyme to elicit said antiproliferative and antimetastatic effects.

The present invention relates generally to methods for controlling life-threatening diseases associated with abnormal cell proliferation and migration, such as cancer, by administering therapeutically effective doses of recombinant human lysozyme (RHL) from genetic engineering to a subject in need thereof to elicit the antimetastatic effect. The invention relates also to methods to elicit said antimetastatic effect by administering any pharmaceutically acceptable addition salt of recombinant human lysozyme or to a mixture thereof.

Human lysozyme is found in human breast milk as well as most epithelial surface secretions including tears, naso-gastric, saliva, bronchial and cervical mucus. In the human body lysozyme acts as a protective barrier against environmental agents and, in doing so, helps prevent infections. Many properties of human lysozyme have been described and already well focused in the international literature, such as antibacterial (it functions by weakening the bonds in the bacterial cell wall), anti-fungal and anti-viral. Lysozyme acts also synergistically with other peptides, such as lactoferrin, to potentiate the activity of both proteins. This peptide plays an important role in the body and significantly enhances human health, such as for instance the gastrointestinal tract health (dietary management of acute diarrhoea), the prevention and treatment of topical infections and many others functions, which have been published in thousands of articles. Few among the most recent publications are listed below: Elison, R. T. et al., J. of Clin. Inv., 88(4): 1080-91 (1991); Humphey B. D. et al., J. of Nutrition, 132: 1214-18 (2002); Huang J. Et al., Mol. Breeding, 10(1-2): 83-94 (2002); Proctor V. A. et al., CRC Critical Reviews in Food Science and Nutrition, 26(4): 359-95 (1988).

Despite these general properties and remarkable functions, the medicinal use of human lysozyme over the past years has been often neglected by the clinicians and by the medical opinion leaders. Another obstacle to its widespread use is the difficulty to obtain human lysozyme in sufficiently large amounts. However, lysozymes from other animal species, even presenting very low homology with human lysozyme, were introduced during the second half of the past century as pharmaceutical products or as nutritional supplements, such as for instance hen egg-white lysozyme (HEL), probably for its large availability and moderate price. The medicinal and nutritional applications of HEL were mainly directed to improving the general health conditions and to ameliorate some negative effects of certain pathological conditions or viral infections, but often with alternate or even contrasting results.

Larger amounts of recombinant proteins, including human lysozyme, completely free from infections or toxic contaminants, with an economically advantaged expression capability of about 1.0% (10 g/Kg) were available during the most recent years from new recombinant technologies. Therefore the present inventors have been increasingly interested to further explore undisclosed methods for new medicinal applications for recombinant human lysozyme from genetic engineering. These technologies have been available since the year 2002, as disclosed in U.S. Pat. No. 6,642,437 producing proteins in plant seeds. Publication WO 02/064750 disclosed the expression of human lysozyme in maturing rice grains transformed using the codon-optimized structural gene for human lysozyme. Publications WO 01/83792 and WO 02/064814 describe food and food additives comprising one or more milk proteins produced in the seeds of a transgenic plant and method of making the same, with the aim to obtain improved infant formula comprising such food supplement composition. More successfully publication WO 05/017168 discloses a mature, transgenic monocot seed that yields, by extracting ground seeds with an aqueous medium, a total soluble protein fraction containing at least 3.0% by total protein weights of a human milk protein, selected from the group consisting of lactoferrin, lysozyme, lactoferricin, lactadherin, kappa-casein, haptocorrin, lactoperoxidase, alpha-lactalbumin, beta-lactoglobulin, alpha-casein, beta-casein and alpha-1-antitrypsin; and a milk-protein composition comprising a total soluble protein fraction in the dried form for use as an additive for an ingestible food or feed.

In addition to the above references, recombinant human lysozyme may be also obtained by genetically-modified host cells, as already known in the art, such as prokaryotic microorganisms like Escherichia coli or Bacillus subtilis or eukaryotic cells like yeast or mammalian cells. Methods for producing said recombinant human lysozyme are described in EP1111054 (equivalent to U.S. Pat. No. 6,660,512), EP1111058 (equivalent to U.S. Pat. No. 6,528,297), EP1111059 (equivalent to U.S. Pat. No. 6,436,688) and EP 1111060 (equivalent to U.S. Pat. No. 6,743,617).

Therefore, nowadays methods are known in the art for producing, isolating and purifying recombinant human lysozyme, using different techniques, but its applications have been until now substantially limited to the nutritional field.

Despite the fact that recombinant human lysozyme has shown to be about 4 times more active per mg for lysing Micrococcus lysodeikticus and Escherichia coli, when compared with HEL, no substantial progress has been registered in medicine, such as additional new methods or uses from those already well known to a skilled artisan. Those well known methods and uses are not the claimed subject matter of the instant invention.

Therefore, preferably the present invention may provide new medicinal uses and therapeutic methods for therapeutically effective doses of medicinal compositions containing RHL from genetic engineering.

This desire has been surprisingly achieved since the applicants have found that RHL remarkably elicits an antimetastatic effect representing a useful method for treating in humans serious diseases associated with abnormal cell proliferation and migration, such as cancer, by administering therapeutically effective doses of said peptide to a subject in need thereof. In fact, the inventors have surprisingly and unexpectedly observed that, in addition to its well known proteolytic antibacterial effect, recombinant human lysozyme experimentally elicits an unforeseen slight antiproliferative activity but in particular an unforeseeable antimetastiatic effect on abnormal cell replication and migration.

### GENERAL

According to one embodiment the present invention there may be provided methods for controlling life-threatening diseases associated with abnormal cell proliferation and migration, by administering therapeutically effective doses of RHL from genetic engineering to a subject in need thereof to elicit a remarkable antimetastatic effect.

In a further embodiment of the present invention there may be provided methods for eliciting said antiproliferative and antimetastatic effects by administering pharmaceutically acceptable addition salt of said peptide or their mixture thereof.

In yet a further embodiment of the present invention there may be provided therapeutically effective doses of RHL to elicit the antimetastatic effect on the abnormal cell replication and migration process, such as cancer, affecting a human subject in need of such medicinal treatment thereof.

In yet another embodiment of the present invention there may be provided therapeutically effective doses of RHL and optionally in association with another active ingredient exerting a complementary, adjuvant or synergic effect on abnormal cell proliferation and/or migration.

In yet another embodiment of the present invention there may provided therapeutically effective dose of RHL formulated with a pharmaceutically acceptable carrier or support suitable to prepare the most adequate and stable medicinal composition to be used for said medicinal purposes.

In yet another embodiment the present invention may relate to the use of RHL for the manufacture of a medicament for the treatment in a human subject of a disease associated with abnormal cell proliferation and migration, such as cancer.

In fact, as will be further disclosed in the following examples, the inventor has unexpectedly and surprisingly found that RHL elicits on abnormal cells, experimentally induced in animal models, not only an inhibition on the proliferation superior to the controls, but also a significant reduction on the cell migration (metastasis).

Thus, the present invention may provide the use of recombinant human lysozyme in the preparation of a medicament for eliciting antiproliferation and/or antimigration effects on abnormal cells. Moreover, the present invention further provides the use of recombinant human lysozyme in the preparation of a medicament for the control of life-threatening diseases associated with abnormal cell proliferation and/or migration, such as cancer metastasis.

The mechanism of action of RHL is still not entirely clear, being still a matter of research by the applicants. One hypothesis is that RHL may stimulate the production or induce higher plasmatic levels of one or more specific antiproliferative cytokines, such as Tumor Necrosis Factor alpha (TNF-α) and/or the like or of one or more interleukines and/or the like, thus inhibiting the replication speed but more particularly the migration of the tumour cell.

The applicants have unexpectedly and surprisingly found that the oral administration of therapeutically effective doses of RHL may positively elicit a significant plasmatic increase of free circulating TNF-α and that to this effect could be possibly due the specific inhibition on abnormal cell proliferation. Particularly when at the initial proliferative stage, as in the case of an experimentally induced cancer implant, the authors have observed such unexpected activity, which is not appreciably present in the group of animals comparatively treated with HEL.

Another hypothesis of the applicants is that oral administration of RHL may stimulate in a certain extent the Toll-Like Receptors (TLR), recently described in the most advanced medical publications. In fact, nine receptors, with different specific and mixed functions, have been recently found and described: Napolitani G. et al., Nat. Immunol., 6(8): 769-76 (2005), Bottino C. et al., Mol. Immunol., 41:569-75 (2004); Takeda K. Et al., J. Derm. Sc. (Review), 34: 73-82 (2004); Vogel S, N. et al., Mol. Interventions. (Review), 3 (8): 466-477 (2003). Therefore RHL or its fragments or bacterial peptidoglycans or viral debris may stimulate directly the specific cancer receptors or indirectly through the other receptors more specific for bacterial and viral infections.

Surprisingly the applicants have also noted that the oral administration of RHL also decreases the plasmatic levels of some aspecific markers, such as sialic acid (total), prolactin and leptin.

The present invention may also provide therapeutically effective doses of RHL or its pharmaceutically acceptable salts in a pharmaceutical composition or medicament. The medicament containing RHL may be any preparations suitable for oral route, such as tablets, with prompt or sustained release, capsules, pills, granules, powder, syrup, suspension, emulsion and the like, all prepared according to general techniques well known to a skilled artisan. Therefore said oral compositions of RHL may optionally additionally contain one or more pharmaceutically acceptable compounds or materials such as diluents, fillers, lubricants, excipients, solvents, binders, stabilizers, thickening agents, and the like.

Diluents that may be used in the compositions of the compounds of the invention include but are not limited to dicalcium phosphate, calcium sulphate, lactose, microcrystalline cellulose, kaolin, mannitol, sucrose, dextrose, sodium chloride, starch and for prolonged release tablets the hydroxypropyl methylcellulose (HPMC), shellac, and the like.

Binders that may be used in the compositions for wet granulation include but are not limited to starch, gelatine, natural and synthetic gums, povidone, sodium alginate, polyethylene glycols, and the like and for direct compression include but are not limited to microcrystalline cellulose, spray dried and anhydrous lactose, starch, di-calcium phosphate, and the like.

Fillers such as sucrose, glucose, dextrose and lactose, and the like may be also used. Natural and synthetic gums that may be used in the compositions include but are not limited to sodium alginate, magnesium aluminium silicate, carboxymethyl cellulose, methyl cellulose, povidone.

Stabilizers that may be used include but are not limited to polysaccharides such as acacia gum, agar, alginic acid, guar gum and tragacanth gum, and disintegrants include croscarmellose sodium type A and other starches and cellulose water soluble derivatives, and the like.

Lubricants, glidants and antiadherents that may be used include but are not limited to metallic stearates (magnesium, calcium and zinc), stearic acid, hydrogenated vegetable oils, talc, corn starch, polyethylene glycols, microfine silicas, and the like. Surfactants like sodium lauryl sulfate and dioctyl sodium sulfosuccinate (DDS) may be also used.

The oral dose unit preferably contains from 20 to 80% of RHL, more preferably from 40% to 60% of the active ingredient. RHL can be used in a substantially similar manner to other known anti-tumour agents for treating various pathological conditions. For recombinant human lysozyme, the dose to be administered, the dosage frequency, the total daily dose, the length of treatment can significantly vary for each subject, in relation to the chosen route of administration, type of tumour, age and body weight of the recipient, excretion rate, drug combination, and general conditions of the patient undergoing therapy.

Accordingly, the dosage to be administered is not subject to definite bounds, but preferably it will be an effective amount to achieve the desired pharmacological and therapeutic effects.

Accordingly, optimal therapeutic concentrations of RHL will be best determined and adjusted through routine clinical experimentation in patients suffering from different diseases to be treated. In fact, an oncologist skilled in the art of cancer treatment will be able to ascertain, without undue experimentation, appropriate protocols for the effective administration of RHL, such as by referring to the earlier published studies on compounds found to have anti-tumour and antimetastatic properties, and then to adjust the dosage regimen in relation to the achieved therapeutic results. Nevertheless the following dosage and regimen are tentatively reported hereby as a non-binding guidance.

The dosages and the dosage regimen in which RHL or its pharmaceutically acceptable salt or a mixture thereof is administered will vary according to the dosage form, mode of administration, the condition being treated and particulars of the subject being treated, and the like.

According to the present invention RHL may be preferably used orally. The therapeutically effective doses of RHL may suitably vary and be administered at the rate of from 5 mg to 200 mg per day per Kg body weight, preferably from 15 to 100 mg per day per Kg of body weight, more preferably from 25 to 50 mg per day per Kg of body weight. The required therapeutically effective daily dose can be administered in one or more portions, according to the available pharmaceutical dose unit. If required from the severity of the disease, the RHL may also be administered parenterally. In that case, RHL is generally administered at the variable dosage regimen of 2 mg to 100 mg per day per Kg of body weight, preferably of 5 to 50 mg per day per Kg of body weight, more preferably from 10 to 20 mg per day per Kg of body weight. The parenteral daily dose can be also administered in one or more portions.

A composition containing RHL is suitably dispensed in unitary pharmaceutical dose form to simplify its administration and to achieve uniformity of the dispensed dosage. Accordingly, each solid dose unit for oral administration may contain a quantity of RHL varying from 50 mg to 1000 mg, preferably from 200 to 500 mg.

In case of oral liquid forms, the above dose units are dissolved in a volume of solvent in multiples of 1.0 ml, such as 5.0 or 10.0 ml or multiples thereof. The volumes to be administered for each dose may be then exactly determined by using currently available devices.

The parenteral dose unit of RHL may contain a quantity of 100 mg to 1500 mg, preferably of 200 mg to 1000 mg. The parenteral dose unit may contain said RHL already dissolved or as lyophilized powder, to be dissolved at the time of use in the prescribed amount of a suitable solvent.

The above pharmaceutical unitary doses of RHL are produced with the conventional techniques already known to a skilled artisan and fully described in specialized publications, like “Remington: The Science and Practice of Pharmacy”, 20.th Edition, 2000.

Because of the experimentally proved inhibitory activity on cell proliferation and migration, the RHL is particularly suitable for use in the preparation of a pharmaceutical composition for treating a related variety of diseases in different conditions. In this regard, “treatment” or “treating” include both therapeutic and prophylactic treatments. Accordingly, RHL may be used to prepare a pharmaceutical composition for treating at very early stages of a disease, or even before early onset, or after significant progression, including metastases. The terms “treatment” and “treating” are used to designate in particular a reduction of the burden in a patient, such as a reduction in cell proliferation rate, a destruction of diseased proliferative cells, a reduction of tumour mass or tumour size, a delaying of tumour progression, a reduction of number and size of metastases, as well as a complete tumour suppression.

Typical examples of diseases associated with abnormal cell proliferation and migration include cancer. The use of recombinant human lysozyme is particularly suited for treating cancer, such as solid tumours or lymphoid tumours. Specific examples of the above pathological conditions include breast cancer, ovarian cancer, prostate cancer, bladder cancer, lung cancer, head and neck cancer, non-Hodgkin's lymphoma, melanoma, colon cancer, pancreas and liver cancers, and the like and their metastases thereof.

The invention is further described for a better understanding in the following Examples, which shall not be considered limitative of the subject matter and of claims of the instant invention.

### EXAMPLE 1

Effects on tumour growth and metastases development of experimental carcinoma of orally administered recombinant human lysozyme (RHL) in mouse.

### a) State of the Art

A number of scientific evidences are supporting the modulation of the immune response of GALT (Gut Associated Lymphoid Tissues) and also the hypothesis involving cytokine-related immunological mechanisms of activation of some recently discovered receptors, such as the Toll-Like Receptors (TLR's) of lymphocytes (Li L., Inflammation & Allergy, 2004, 3: 81-86; Vogel S, N. et al., Molecular Interventions, 2003, 3: 466-477) may represent a suitable system to treat systemic pathologies related to abnormal cell proliferation and migration.

### b) Aim of the Study

The purpose of the present research study is to determine, in vivo, the effects of RHL of the invention on the tumour growth and metastases development in mouse model of experimental carcinoma. RHL was administered orally for 14 consecutive days, admixed to the powdered food. The effect of RHL was compared to that of commercially available hen egg-white lysozyme (HEL).

### c) Drugs and Dose Levels

RHL (Lot P-0056) used for the experimental study was purchased from Ventria Bioscience Inc. (Sacramento, Calif., U.S.A.). HEL, extracted from hen egg-white, was used as a reference product of the research, and is currently available on the market (SPA Società Prodotti Antibiotici S.p.A., Milano, Italy).

The research study in mouse was performed on behalf and in accordance to Applicant's instructions by an independent and specialized Contract Research Organization (CRO).

RHL (Lot P-0056) was administered at three dose levels: 25 mg/kg/day (RHL25), 50 mg/kg/day (RHL50) and 100 mg/kg/day (RHL100), while HEL was administered at the dose level of 100 mg/kg/day (HEL100). All substances were added to the daily powdered food for 14 consecutive days. Food consumption was calculated separately and the medicated food was renewed daily. The amount of food discarded by the animals influenced the daily dosage by less than 10.0%.

### d) Animal Model

CBA female mice, of 17-18 g of body weight, used for the test were sourced by HARLAN Italy Srl, S. Pietro al Natisone (UD), Italy. The animals were allowed to be fed with powdered food for 7 days before the experiment. The animals were maintained under conventional procedures (with drinkable tape-water ad libitum) in suitable animal house facilities at the Department of Biomedical Sciences of the University of Trieste, which were partially hired by the appointed CRO.

### e) Solid Tumour Model

The murine mammary carcinoma line (MCa) used in the present work derived from a spontaneous solid tumour of CBA female mice, subsequently isolated and stabilised at Rudjer Boskovic Institute, Zagreb, Croatia. The tumour cell line in use at Callerio Foundation Onlus, Trieste, Italy was stored in cryogenic tanks with liquid nitrogen, in cryogenic vials containing 10⁷ viable cells of a single-cell suspension obtained from primary tumours at the third transplant generation.

The tumour was grown in vivo by implanting intramuscularly (i.m.) into the calf of the left hind leg of CBA female mice the content of one vial of cells of MCa taken from the cryogenic tank. The tumour of two mice was harvested from the legs after 14 days (about 2 g of tumour mass) and a single-cell suspension was prepared and re-implanted into 4 CBA female mice which constitute the donors for tumour propagation for the experimental purposes. The tumour, harvested from mice under sterile conditions (animals were killed by cervical dislocation, the area of tumour growth was disinfected with an appropriate solution and the animals were placed under a sterile box), was pooled, minced with scissors, re-diluted with Dulbecco's phosphate buffered saline (pH 7.4) containing Ca²⁺ and Mg²⁺ (PBS) and filtered with a double layer of sterile gauze to remove tissue debris. The cell suspension was then centrifuged at 250×g at 0-4° C. during 10 min; the supernatant was discarded while the pellet was re-suspended in an equal volume of PBS, and cell concentration and viability were determined by the trypan blue exclusion test with a Burker's camera.

Viable cells were finally diluted in order to have 1.0×10⁶ cells/0.05 ml PBS. For the experimental purposes, 110 CBA mice were implanted i.m. into the calf of the left hind leg with 10⁶ viable MCa carcinoma cells of a cell suspension obtained from the primary tumour harvested from the 4 CBA donors.

### f) Experimental Protocol

Primary tumours were implanted i.m. on Day 0 (Feb. 3, 2005). Mice were then randomized into the test groups (Table 1). The protocol was constituted of four arms of treatment and one arm of control.

Primary tumour growth was evaluated on Day 7 (Feb. 10, 2005), on Day 9 (Feb. 12, 2005), on Day 11 (Feb. 14, 2005), on Day 13 (Feb. 16, 2005), on Day 15 (Feb. 18, 2005) and on Day 18 (Feb. 21, 2005). Measurements of the two orthogonal axes of the tumour were done by means of a calliper. Primary tumour weight was calculated by the formula: (π/6)×a²×b, where a and b are the minor and major perpendicular axis respectively and subtracting to the result the weight of the tumour-free leg hind (that correspond to about 100 mg).

Lung metastases number and weight were evaluated on Day 20 (Feb. 23, 2005) after killing the animals by cervical dislocation. Lung metastases were counted and measured on Day 20 (Feb. 23, 2005) by means of a low-power stereo microscope equipped with a graduated grid. Metastases' weight was determined by the formula: (π/6)×a²×b, where a and b are the minor and major orthogonal axis respectively. Spleens weight of three random chosen animals per group was taken on Day 20 (Feb. 23, 2005) after killing the animals by cervical dislocation. Cages containing animals were controlled daily to prevent suffering of tumour-bearing mice.

TABLE 1
Design of the treatment arms of experimental protocol.

| | Lysozyme dose |
|---|---|
| Groups | mg/kg/day |
| Controls | — |
| HEL 100 | 100 |
| RHL 100 | 100 |
| RHL 50 | 50 |
| RHL 25 | 25 |

### g) Tabulation and Results

The determined results of the considered parameters are summarized in the following Tables:

TABLE 2
Animal deaths during treatment (Day 0-Day 20).

| | Feb. | Feb. | Feb. | Feb. | Feb. | Feb. | Feb. | |
|---|---|---|---|---|---|---|---|---|
| | 3^{rd} | 18^{th} | 19^{th} | 20^{th} | 21^{st} | 22^{nd} | 23^{rd} | |
| | 2005 | 2005 | 2005 | 2005 | 2005 | 2005 | 2005 | |
| | Progressive day of treatment | | | | | | | Total |
| Groups | 0 | 15 | 16 | 17 | 18 | 19 | 20 | deaths |
| Controls | 0/10 | 1/10 | 0/9 | 0/9 | 0/9 | 1/9 | 1/8 | 3/10 |
| HEL 100 | 0/9 | 1/9 | 1/8 | 0/7 | 1/7 | 1/6 | 1/5 | 5/9 |
| RHL 100 | 0/9 | 0/9 | 0/9 | 1/9 | 1/8 | 0/7 | 0/7 | 2/9 |
| RHL 50 | 0/8 | 0/8 | 0/8 | 0/8 | 1/8 | 0/7 | 1/6 | 2/8 |
| RHL 25 | 0/8 | 0/8 | 0/8 | 0/8 | 0/8 | 1/8 | 0/7 | 1/8 |

The number of living animals per each group was indicated, starting from the day of tumour implant (Day 0) up to the day of sacrifice (Day 20). The total number of animals that died before the end of the experiment was 13/44.

TABLE 3
Body weight variations (in g) between Day 0 (day of tumour implant)
and Day 20 (end of the experiment and metastases evaluation).

| | Body weight variation (in g) during treatment period | | |
|---|---|---|---|
| | Day 0 | Day 20 | Variation (% of the |
| Groups | mean ± S.E. | mean ± S.E. | respective Controls) |
| Controls | 17.6 ± 0.3 | 18.3 ± 0.3 | — |
| HEL 100 | 18.3 ± 0.4 | 17.5 ± 0.7 | −8.0 vs Controls |
| RHL 100 | 17.9 ± 0.3 | 18.5 ± 0.5 | −0.6 vs Controls |
| RHL 50 | 18.4 ± 0.5 | 19.1 ± 0.7 | −0.2 vs Controls |
| RHL 25 | 17.9 ± 0.5 | 18.3 ± 0.6 | −1.7 vs Controls |

Weight variations were calculated according to the formula

{[(Group X m.w. on Day 20/Group X m.w. on Day 0)/(Group Y m.w. on Day 20/Group Y m.w. on Day 0)]*100}−100

m.w.=mean weight

TABLE 4
Effect on primary tumour growth (in mg).

| | Primary tumour mass (mg) - mean ± S.E. | | | | | |
|---|---|---|---|---|---|---|
| | Date of treatment | | | | | |
| | Feb. | Feb. | Feb. | Feb. | Feb. | Feb. |
| | 10^{th} | 12^{th} | 14^{th} | 16^{th} | 18^{th} | 21^{st} |
| | 2005 | 2005 | 2005 | 2005 | 2005 | 2005 |
| | Progressive day of treatment | | | | | |
| Groups | 7 | 9 | 11 | 13 | 15 | 18 |
| Controls | 246 ± 15 | 350 ± 15 | 642 ± 43 | 1043 ± 63 | 1519 ± 85 | 2682 ± 126 |
| HEL 100 | 231 ± 24 | 338 ± 21 | 591 ± 35 | 1062 ± 104 | 1325 ± 189 | 2133 ± 130 |
| RHL 100 | 251 ± 21 | 349 ± 40 | 669 ± 84 | 1140 ± 133 | 1462 ± 168 | 2643 ± 145 |
| RHL 50 | 287 ± 27 | 355 ± 44 | 692 ± 101 | 1110 ± 142 | 1473 ± 213 | 2404 ± 147 |
| RHL 25 | 304 ± 19 | 374 ± 39 | 611 ± 70 | 1136 ± 129 | 1533 ± 103 | 2344 ± 190 |

TABLE 5
Effect on lung metastases formation.

| | Lung metastases | | |
|---|---|---|---|
| | Number/mouse | Weight (mg)mouse | Metastases-free |
| Groups | mean ± S.E. | mean ± S.E. | animals |
| Controls | 38 ± 3 | 69.86 ± 14.75 | 2/7 |
| HEL 100 | 31 ± 8 | 41.76 ± 28.69 | 0/4 |
| RHL 100 | 22 ± 4 | 8.73 ± 3.71 | 1/7 |
| RHL 50 | 30 ± 6 | 8.90 ± 2.26 | 2/6 |
| RHL 25 | 29 ± 5 | 27.06 ± 9.12 | 0/7 |

TABLE 6
Spleen weights (in g) at the day of sacrifice (Day 20).

| | Spleen weight | % of body |
|---|---|---|
| Groups | mean ± S.E. | weight (*100) |
| Controls | 0.28 ± 0.01 | 1.55 |
| HEL 100 | 0.34 ± 0.07 | 1.94 |
| RHL 100 | 0.34 ± 0.02 | 1.86 |
| RHL 50 | 0.39 ± 0.04 | 2.02 |
| RHL 25 | 0.36 ± 0.02 | 1.97 |

### h) Result Summary and Conclusions

### h.1) Effects on Primary Tumour Growth

h.1.1) Results Summary
The oral treatment of mammary carcinoma MCa tumour-bearing mice with recombinant human lysozyme (RHL), at the doses of 100, 50 and 25 mg/kg/day, is free of significant effects on primary tumour growth.
Also hen egg-white lysozyme (HEL) at the dose of 100 mg/kg/day is not effective on primary tumour growth (p<0.05 versus untreated controls).

### h.2) Effects on Metastases Development

h.2.1) Results Summary
RHL, at the doses of 100, 50 and 25 mg/kg/day, significantly reduces the mean overall lung metastasis mass (p<0.05-p<0.01 versus untreated controls) in a dose-dependent way.
HEL, at 100 mg/kg/day, is ineffective on both metastases growth and number.

### h.3) Conclusions

RHL did not show anti-tumour effects on MCa mammary carcinoma at the primary tumour site.
RHL remarkably reduces the overall lung metastatic mass.
The effectiveness RHL on metastases development is dependent on the administered dose in the range of doses from 25 to 100 mg/kg/day.

### EXAMPLE 2

Preparation of 100,000 tablets containing 500 mg of recombinant human lysozyme.

Each tablet containing:

| Ingredients | Quantity (per tablet) | |
|---|---|---|
| Recombinant human lysozyme | 500 | mg |
| Microcrystalline cellulose | 250 | mg |
| Magnesium stearate | 25 | mg |
| Silicon dioxide | 15 | mg |
| Total weight | 790 | mg |

The manufacturing process was carried out by direct compression of the powder mixture through the steps already known to a skilled artisan to prepare tablets, using suitable rooms and equipments for this type of production.

The necessary ingredients to manufacture 100,000 tablets of RHL were weighed and processed for direct compression according to the techniques well known to a skilled artisan to yield the desired tablets. 96,280 biconvex tablets were obtained. Yield: 96.28%.

### EXAMPLE 3

Preparation of 1,000 injectable ampoules (10.0 ml) containing 300 mg/ml of recombinant human lysozyme (as hydrochloride).

Each ampoule of 10.0 ml containing:

| Ingredients | Quantity (per vial of 10.0 ml) | |
|---|---|---|
| Recombinant human lysozyme | 3000 | mg |
| (as hydrochloride) | | |
| Sodium chloride | 800 | mg |
| Water for parenteral preparations q.s. to | 10.0 | ml |

The manufacturing process was carried out through the steps already known to a skilled artisan to prepare ampoules of parenteral use, using suitable rooms and equipments for this type of production.

The necessary ingredients to manufacture 1,000 ampoules of 10.0 ml were weighed and processed by industrial process according to the techniques well known to a skilled artisan to yield the desired ampoules. 834 ampoules of 10.0 ml of solution were obtained.

Yield: 83.4%.

Although the foregoing invention has been described in some detail by way of illustration and examples, for purposes of clarity and of understanding, it will be obvious that certain changes and modifications may be practised within the scope of the appended claims.

1. The use of recombinant human lysozyme in the preparation of a medicament for eliciting antiproliferation and/or antimigration effects in abnormal cells.

2. The use of recombinant human lysozyme in the preparation of a medicament according to claim 1, for the control of life-threatening diseases associated with abnormal cell proliferation and/or migration, such as cancer metastasis.

3. The use of recombinant human lysozyme in the preparation of a medicament according to claim 1, wherein the recombinant human lysozyme is obtained either by a DNA recombinant technique for producing proteins in plant seeds, such as maturing rice grains transformed using codon-optimized structural gene for human lysozyme, or by a fermentation technique where a selected microorganism has been genetically modified with a gene construct to yield human lysozyme.

4. The use of recombinant human lysozyme in the preparation of a medicament according to claim 1, wherein the recombinant human lysozyme is obtained by genetically-modified host cells, such as prokaryotic microorganisms or eukaryotic cells like yeast or mammalian cells.

5. The use of recombinant human lysozyme according to claim 1, wherein recombinant human lysozyme represents also its pharmaceutically acceptable salt or a mixture thereof.

6. The use of recombinant human lysozyme according to claim 1 wherein the medicament comprises a therapeutically effective amount of the recombinant human lysozyme, and wherein the therapeutically effective dose of recombinant human lysozyme for oral administration varies from 5 mg to 200 mg/Kg body weight per day, preferably from 15 mg to 100 mg/Kg body weight per day.

7. The use of recombinant human lysozyme according to claim 6, wherein the therapeutically effective dose of recombinant human lysozyme more preferably varies from 25 mg to 50 mg/Kg per day.

8. The use of recombinant human lysozyme according to claim 1 wherein the medicament comprises a therapeutically effective amount of recombinant human lysozyme, and wherein the therapeutically effective dose of recombinant human lysozyme for parenteral administration varies from 2 mg to 100 mg/Kg body weight per day, preferably from 5 mg to 50 mg/Kg body weight per day.

9. The use of recombinant human lysozyme according to claim 8, wherein the therapeutically effective dose of recombinant human lysozyme more preferably varies from 10 mg to 20 mg/Kg per day.

10. The use of recombinant human lysozyme according to claim 1, wherein the medicament further optionally comprises one or more pharmaceutically acceptable compounds or materials.

11. The use of recombinant human lysozyme according to claim 10, wherein the one or more optional pharmaceutically acceptable compounds or materials are active ingredients exerting a complementary, adjuvant and/or synergic effect on abnormal cells, and/or one or more pharmaceutically acceptable vehicles and/or supports suitable for oral and parenteral administration.

12. A method to elicit antiproliferation and antimigration effect on abnormal cells, such as cancer metastasis, by administering a therapeutically effective amount of recombinant human lysozyme to a human subject in need of such medicinal treatment thereof.

13. A method to control life-threatening diseases associated with abnormal cell proliferation and migration, such as cancer metastasis, by administering therapeutically effective doses of recombinant human lysozyme to a subject in need of such medicinal treatment thereof.
